SEQUENCE LISTING (28 sequences)

SEQ ID NO: 1 - protein, 233 aa, Homo sapiens
  SOURCE 1..233: /mol_type=“protein” /organism=“Homo sapiens”
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 5 aa, Homo sapiens
  SOURCE 1..5: /mol_type=“protein” /organism=“Homo sapiens”
PSDKP

SEQ ID NO: 3 - protein, 5 aa, Homo sapiens
  SOURCE 1..5: /mol_type=“protein” /organism=“Homo sapiens”
VANPQ

SEQ ID NO: 4 - protein, 135 aa, Homo sapiens
  SOURCE 1..135: /mol_type=“protein” /organism=“Homo sapiens”
RDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGV

SEQ ID NO: 5 - protein, 17 aa, Homo sapiens
  SOURCE 1..17: /mol_type=“protein” /organism=“Homo sapiens”
VRSSSRTPSDKPVAHVV

SEQ ID NO: 6 - protein, 23 aa, Homo sapiens
  SOURCE 1..23: /mol_type=“protein” /organism=“Homo sapiens”
FLSLFSFLIVAGATTLFCLLHFG

SEQ ID NO: 7 - protein, 18 aa, Homo sapiens
  SOURCE 1..18: /mol_type=“protein” /organism=“Homo sapiens”
IGPQREEFPRDLSLISPL

SEQ ID NO: 8 - protein, 38 aa, Homo sapiens
  SOURCE 1..38: /mol_type=“protein” /organism=“Homo sapiens”
QLVVPSEGLYLIYSQVLFKGQGCPSTHVLLTHTISRIA

SEQ ID NO: 9 - protein, 15 aa, Homo sapiens
  SOURCE 1..15: /mol_type=“protein” /organism=“Homo sapiens”
PCQRETPEGAEAKPW

SEQ ID NO: 10 - protein, 41 aa, Homo sapiens
  SOURCE 1..41: /mol_type=“protein” /organism=“Homo sapiens”
SMIRDVELAEEALPKKTGGPQGSRRCLFLSLFSFLIVAGAT

SEQ ID NO: 11 - protein, 35 aa, Homo sapiens
  SOURCE 1..35: /mol_type=“protein” /organism=“Homo sapiens”
VLLTHTISRIAVSYQTKVNLLSAIKSPCQRETPEG

SEQ ID NO: 12 - protein, 157 aa, Homo sapiens [sequence omitted]

SEQ ID NO: 13 - protein, 189 aa, Homo sapiens
  SOURCE 1..189: /mol_type=“protein” /organism=“Homo sapiens”
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 14 - protein, 188 aa, Homo sapiens [sequence omitted]

SEQ ID NO: 15 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 16 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 17 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 18 - protein, 189 aa, Homo sapiens
  SOURCE 1..189: /mol_type=“protein” /organism=“Homo sapiens”
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 19 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 20 - protein, 189 aa, Homo sapiens
  SOURCE 1..189: /mol_type=“protein” /organism=“Homo sapiens”
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 21 - protein, 189 aa, Homo sapiens
  SOURCE 1..189: /mol_type=“protein” /organism=“Homo sapiens”
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 22 - protein, 189 aa, Homo sapiens
  SOURCE 1..189: /mol_type=“protein” /organism=“Homo sapiens”
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 23 - protein, 235 aa, Homo sapiens
  SOURCE 1..235: /mol_type=“protein” /organism=“Homo sapiens”
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 24 - protein, 20 aa, Homo sapiens
  SOURCE 1..20: /mol_type=“protein” /organism=“Homo sapiens”
PLALLLHAARPSQFRVSPLD

SEQ ID NO: 25 - protein, 20 aa, Homo sapiens
  SOURCE 1..20: /mol_type=“protein” /organism=“Homo sapiens”
AEGLDTQRFSGKRLGDTFVL

SEQ ID NO: 26 - protein, 20 aa, Homo sapiens
  SOURCE 1..20: /mol_type=“protein” /organism=“Homo sapiens”
SIMYFSHFVPVFLPAKPTTT

SEQ ID NO: 27 - protein, 10 aa, Homo sapiens
  SOURCE 1..10: /mol_type=“protein” /organism=“Homo sapiens”
VITLYCNHRN

SEQ ID NO: 28 - protein, 15 aa, Homo sapiens
  SOURCE 1..15: /mol_type=“protein” /organism=“Homo sapiens”
VVKSGDKPSLSARYV

(19) United States
(12) Patent Application Publication
(10) Pub. No.: US 2013/0224219 A1
(43) Pub. Date: Aug. 29, 2013

(54) DRUG AND METHOD FOR THE PROPHYLAXIS OF HIV INFECTION AND FOR THE PROPHYLAXIS AND TREATMENT OF DISEASES CAUSED BY OR ASSOCIATED WITH HIV, INCLUDING AIDS

(71) Applicants: Oleg Iliich Epshtein, Moscow (RU); Sergei Alexandrovich Tarasov, Golitsyno (RU)

(72) Inventors: Oleg Iliich Epshtein, Moscow (RU); Sergei Alexandrovich Tarasov, Golitsyno (RU)

(73) Assignee: Oleg Iliich Epshtein, Moscow (RU)

(21) Appl. No.: 13/814,659

(22) PCT Filed: Jul. 15, 2011
(86) PCT No.: PCT/RU2011/000523

§ 371 Date: Apr. 17, 2013

(30) Foreign Application Priority Data
Aug. 6, 2010 (RU) 2010133045

(51) Int. Cl. C07K 16/28

(52) U.S. Cl. CPC C07K 16/28
USPC 424/158.1; 530/389.6; 530/389.2; 424/173.1

(57) ABSTRACT

The drug for the prophylaxis of HIV infection and for the prophylaxis and treatment of diseases caused by or associated with HIV, including AIDS, comprises an activated, potentiated form of antibodies to a protein or peptide of the immune system which interacts with the HIV or has a content and/or functional activity which changes in connection with an HIV infection. Furthermore, in the method for the prophylaxis of HIV infection and for the prophylaxis and treatment of diseases caused by or associated with HIV, including AIDS, use is made of an activated, potentiated form of antibodies to an antigen, namely a protein or peptide of the immune system, which interacts with the HIV or has a content and/or functional activity which changes in connection with an HIV infection

### FIELD OF INVENTION

The invention relates to the field of medicine and may be use for the effective prophylaxis of HIV, prophylaxis and treatment of diseases caused by HIV or associated with HIV, including AIDS.

### BACKGROUND

The state of the art provides a medicinal agent for treatment of infectious disease including diseases of viral ethiology, on the basis of activated form of ultra low dose of antibodies to interferon (RU 2192888 C1 A61K39/395, Nov. 20, 2002. However, this known medicinal agent cannot be effective for the prophylaxis of HIV as well as for the prophylaxis and treatment of broad spectrum of diseases caused by HIV or associated with HIV, including AIDS.

### DESCRIPTION OF THE INVENTION

The invention is related to a complex medicinal agent that does not possess marked side effects which is useful as effective prophylaxis agent for HIV as well as for prophylaxis and effective treatment of disease caused by HIV or associated with HIV, including infections and parasitic disease, malignant tumors and AIDS in persons infected by HIV.

The solution to the problem is provided by a medicinal agent for prophylaxis of HIV, prophylaxis and treatment of diseases caused by HIV or associated with HIV, including AIDS, which contains activated-potentiated forms of antibodies to antigen—a protein or a peptide of the immune system or, primarily, produced by the immune system, which interacts with HIV or content and/or functional activity of which are altered in the presence of HIV.

It is contemplated that the activated-potentiated form of antibodies, primarily, to the solved antigen (or soluble antigen, i.e., antigen not bound to the outer membrane of the immune cells) can be used.

Cytokines (except for gamma-interferon) can be used as dissolve antigen.

In addition, it is possible to use the activated-potentiated form of antibodies primarily to antigen connected with the outer membrane of cells of the immune system.

Under these circumstances, as an antigen bound with the outer membrane of cells of the immune system, the receptors of immunocompetent cells are used.

The activated-potentiated form of antibodies to solved antigens or the activated-potentiated form of antibodies to antigens bound to the outer membrane of the immune cells are used in the form of activated-potentiated aqueous or aqueous-alcoholic solution the activity of which is based on the process of serial multiple consecutive dilutions of the stock (initial) solution of antibodies in aqueous or aqueous-alcoholic solvent combined with the external mechanical impact—vertical shaking after each dilution.

The claimed medicinal agent can be produced in solid dosage form as a pharmaceutical composition that contains technologically necessary (effective) amount of neutral carrier impreganted with mixture of aqueous or aqueous-alcoholic solutions of the activated-potentiated form of antibodies to dissolved antigens or the activated-potentiated form of antibodies to antigens bound to the outer membrane of the immune cells and pharmaceutically acceptable excipients including, for example, lactose, microcrystal cellulose, and magnesium stearate.

Aqueous and aqueous-alcoholic solutions of the activated-potentiated forms of antibodies to dissolved antigens or antigens bound to the outer membrane of the immune cells can be obtained by multiple consecutive dilutions of the stock (initial) solution of antibodies combined with the external mechanical impact—vertical shaking of each dilution; the stock solution concentration is 0.5-5.0 mg/mL.

Activated-potentiated form of the antibodies can be used in the form of mixture of different, predominantly centesimal, dilutions by the homeopathic technology.

The solution of the problem is also carried out by the fact that in the context of the method of prophylaxis of HIV, prophylaxis and treatment of diseases caused by HIV or associated with HIV, including AIDS, in accordance with the invention there is used an activated-potentiated form of antibody to antigen—protein or peptide of immune system or primarily expressed by the immune system, which interacts with HIV or the content and/or functional activity of which changes as a result of HIV infection.

The activated-potentiated form of antibodies to dissolved antigens or the activated-potentiated form of antibodies to antigens bound to the outer membrane of the immune cells are used in the form of activated-potentiated aqueous or aqueous-alcoholic solution of each component the activity of which is based on the process of multiple dilution of the stock (initial) solution of antibodies in aqueous or aqueous-alcoholic solvent combined with the external mechanical impact—vertical shaking of each dilution.

Preferably, aqueous and aqueous-alcoholic solutions of the activated-potentiated forms of antibodies to solved antigens or antigens bound to the outer membrane of the immune cells are obtained by serial multiple consecutive dilution of the stock (initial) solution of antibodies combined with the external mechanical impact—vertical shaking of each dilution; the stock solution concentration is 0.5-5.0 mg/mL.

According to the invention, the activated-potentiated form is a form of antibodies prepared in accordance with homeopathic technology of potentiation by the multiple consecutive dilution of the stock (initial) solution of antibodies combined with the external mechanical impact—vertical shaking after each dilution which possesses activity in pharmacological models and/or clinical methods of prophylaxis of HIV, prophylaxis and treatment of diseases caused by HIV or associated with HIV, including AIDS.

Proposed use of the activated-potentiated form of antibodies to solved antigens (for example, to tumor necrosis factor alpha or human alpha interferon) or antigens bound to the outer membrane of the immune cells (for example, to CD8 receptor) leads to the unexpected therapeutic effect that consists in higher efficacy of the medicinal agent in prevention of HIV infection as well as in prophylaxis and treatment of diseases caused by HIV or HIV-associated diseases including AIDS.

It is shown experimentally that the claimed medicinal agent is characterized by high prophylaxis efficacy towards HIV, preventing infection of cells with the human immunodeficiency virus and its intracellular replication and therefore, can be used for the effective treatment as well as for the prevention of viral diseases prone to chronic course including secondary prevention of HIV-infection.

The claimed medicinal agent may be used in combination with antiretroviral agents including complex agents such as reverse transcriptase inhibitors (for example, zidovudine derivatives) which allows a reduction of dose of antiretroviral agents, while maintaining high efficiency of treatment, increase in the safety of the therapy and reduces adverse events.

### EMBODIMENTS OF INVENTION

The medicinal agent is prepared mainly in the following way.

For preparation of the activated-potentiated form of active components, monoclonal or (predominantly) polyclonal antibodies are used; they may be obtained using known technologies, particularly, techniques described, for example, in Immunological methods, under the editorship of G. Frimel, M., ‘Meditsina’, 1987, p. 9-33 [Russian]; or in the article Laffly E., Sodoyer R. Hum. Antibodies. Monoclonal and recombinant antibodies, 30 years after.—2005—Vol. 14.—N 1-2. P. 33-55.

Monoclonal antibodies are obtained, for example, using hybridoma technology. The initial stage of the process includes immunization based on principles that has already been developed for polyclonal antiserum preparation. Further stages of this process involve obtaining of hybridoma cells producing clones of antibodies of the same specificity. Their isolation is carried out using the same methods as for polyclonal antisera.

Polyclonal antibodies can be obtained by active immunization of animals. For this purpose, using a specially developed scheme, the animals receive a series of injections of the substance required according to the invention—antigen or conjugated antigen (protein or peptide of the immune system or, primarily expressed by the immune system which interacts with HIV or the content and/or the functional activity which changes as a result of HIV infection). As a result of this procedure a monospecific antiserum is obtained which is used for producing the activated-potentiated form. If necessary, a purification of antibodies present in the antiserum is performed, for example, using methods of affinity chromatography, salt fractionation, or ion-exchange chromatography.

For example, for production of the claimed medicinal agent, polyclonal antibodies to tumor necrosis factor alpha can be used, which used as a stock (initial) solution (concentration 0.5-5.0 mg/mL) for further preparation of the activated-potentiated form.

It is preferable to use polyclonal antibodies for preparation of the claimed medicinal agent; they can be obtained using immunization of rabbits as follows.

For example, polyclonal antibodies to tumor necrosis factor alpha (TNF-α) can be obtained using whole molecules of TNF-α with the following sequence:

| | |
|---|---|
| 1 | MSTESMIRDV ELAEEALPKK TGGPQGSRRC LFLSLFSFLI |
| | VAGATTLFCL LHFGVIGPQR |
| 61 | EEFPRDLSLI SPLAQAVRSS SRTPSDKPVA HVVANPQAEG |
| | QLQWLNRRAN ALLANGVELR |
| 121 | DNQLVVPSEG LYLIYSQVLF KGQGCPSTHV LLTHTISRIA |
| | VSYQTKVNLL SAIKSPCQRE |
| 181 | TPEGAEAKPW YEPIYLGGVF QLEKGDRLSA EINRPDYLDF |
| | AESGQVYFGIIAL |

The usage of the polypeptide fragment tumor necrosis factor selected, for example, from the following consequences is possible for obtaining of polyclonal antibodies for the tumor necrosis factor-alpha (TNF-α):

84-88:
PSDKP
93-97:
VANPQ
65-199:
RDLSLI SPLAQAVRSS SRTPSDKPVA HVVANPQAEG
QLQWLNRRAN ALLANGVELR DNQLVVPSEG LYLIYSQVLF
KGQGCPSTHV LLTHTISRIA VSYQTKVNLL SAIKSPCQRE
TPEGAEAKPW YEPIYLGGV
77-93:
RSS SRTPSDKPVA HVV
32-54:
IGPQR EEFPRDLSLI SPL
123-160:
QLVVPSEG LYLIYSQVLF KGQGCPSTHV LLTHISRIA
176-190:
PCQRE TPEGAEAKPW
5-45:
SMIRDV ELAEEALPKK TGGPQGSRRC LFLSL
150-184:
V LLTHTISRIA VSYQTKVNLL SAIKSPCQRE TPEG
77-233:
VRSSSRTPSDKPVAHVVANPQAEGQLQWLNRRANALLANGV
ELRDNQLVVPSEGLYLIYSQVLFKGQGCPSTHVLLTHTISRIAVSYQ
TKVNLLSAIKSPCQRETPEGAEAKPWYEPIYLGGVFQLEKGDRLSA
EINRPDYLDFAESGQVYFGIIAL.

Seven-nine days prior to the collection of blood, 1-3 intravenous injections are executed in order are administered to increase the level of the antibodies. During the process of immunization, small samples of blood are taken from the rabbits to evaluate the quantity of the antibodies. The maximum level of the immune response to the administration of antigen is observed 40-60 days after the first injection. After the first cycle of the immunization of the rabbits 30 days are provided to restore their health and repeat the immunization that includes 1-3 intravenous injections. In order to obtain the antiserum the blood is gathered from the immunized rabbits into 50 ml centrifuge tube. Using a wooden spatula the formed clots are eliminated from the walls of the tube and the stick is placed to the clot that is formed at the centre of the tube. The blood is placed to refrigerator (the temperature is 4° C.) overnight. The next day the clot that attached to the spatula is removed and the remaining liquid is centrifuged under 13000 g for 10 minutes. The supernatant (over-sediment liquid) represents antiserum. The obtained antiserum must have yellow color. 20% NaN₃ may be added till the final concentration of 0.02% and it should be kept frozen under the temperature of −20° C. (or without the addition of NaN₃ under the temperature of −70°) before use. The separation of the antibodies from the antiserum to the tumor necrosis factor-alpha is carried out as follows:
1. 10 ml of rabbit antiserum is diluted in 2 times 0.15 M of NaCl, add 6.26 g Na₂SO₄, then mix and incubate for 12-16 hours at 4° C.;
2. The formed sediment is eliminated by the means of centrifuging, diluted in 10 ml of phosphate buffer and then dialyzed to the same buffer during the night under room temperature;
3. After removal of the sediment by centrifugation, the solution is placed to the column with DEAE-cellulose that is equilibrated with phosphate buffer;
4. The fraction of the antibodies is determined using optical density of the eluate at 280 nm.

Purification of antibodies is carried out by affinity chromatography on a column with an antigen, using binding of antibodies to the tumor necrosis factor alpha with an antigen (tumor necrosis factor alpha), bound to the insoluble matrix of the column with the following elution of the antibodies by the concentrated solutions of salt.

The buffer solution of polyclonal antibodies to the tumor necrosis factor alpha obtained by this method which has the concentration of 0-5 0 5.0 mg/ml, preferably 2.0-3.0 mg/ml is used as matrix (initial) solution for the further preparation of activated-potentiated form of the antibodies.

The polyclonal antibodies to the human alpha-interferon are obtained by the above described methodology, using as an immunogen (antigen) for immunization of rabbit adjuvant and the whole molecule of human alpha-interferon in accordance with one of the following sequences:

Human alpha-interferon (subtype 2):
MALTFALLVA LLVLSCKSSC SVGCDLPQTH SLGSRRTLML
LAQMRKISLF SCLKDRHDFG
FPQEEFGNQF QKAETIPVLH EMIQQIFNLF STKDSSAAWD
ETLLDKFYTE LYQQLNDLEA
CVIQGVGVTE TPLMKEDSIL AVRKYFQRIT LYLKEKKYSP
CAWEVVRAEI MRSFSLSTNL
QESLRSKE
Human alpha-interferon (subtype 1/13):
MASPFALLMV LVVLSCKSSC SLGCDLPETH SLDNRRTLML
LAQMSRISPS SCLMDRHDFG
FPQEEFDGNQ FQKAPAISVL HELIQQIFNL FTTKDSSAAW
DEDLLDKFCT ELYQQLNDLE
ACVMQEERVG ETPLMNADSI LAVKKYFRRI TLYLTEKKYS
PCAWEVVRAE IMRSLSLSTN
LQERLRRKE
Human alpha-interferon (subtype 17):
MALSFSLLMA VLVLSYKSIC SLGCDLPQTH SLGNRRALIL
LAQMGRISPF SCLKDRHDFG
LPQEEFDGNQ FQKTQAISVL HEMIQQTFNL FSTEDSSAAW
EQSLLEKFST ELYQQLNNLE
ACVIQEVGME ETPLMNEDSI LAVRKYFQRI TLYLTEKKYS
PCAWEVVRAE IMRSLSFSTN
LQKILRRKD
Human alpha-interferon (subtype 4):
MALSFSLLMA VLVLSYKSIC SLGCDLPQTH SLGNRRALIL
LAQMGRISHF SCLKDRHDFG
FPEEEFDGHQ FQKAQAISVL HEMIQQTFNL FSTEDSSAAW
EQSLLEKFST ELYQQLNDLE
ACVIQEVGVE ETPLMNEDSI LAVRKYFQRI TLYLTEKKYS
PCAWEVVRAE IMRSLSFSTN
LQKRLRRKD
Human alpha-interferon (subtype 8):
MALTFYLLVA LVVLSYKSFS SLGCDLPQTH SLGNRRALIL
LAQMRRISPF SCLKDRHDFE
FPQEEFDDKQ FQKAQAISVL HEMIQQTFNL FSTKDSSAAL
DETLLDEFYI ELDQQLNDLE
SCVMQEVGVI ESPLMYEDSI LAVRKYFQRI TLYLTEKKYS
SCAWEVVRAE IMRSFSLSIN
LQKRLKSKE
Human alpha-interferon (subtype 7):
MARSFSLLMV VLVLSYKSIC SLGCDLPQTH SLRNRRALIL
LAQMGRISPF SCLKDRHEFR
FPEEEFDGHQ FQKTQAISVL HEMIQQTFNL FSTEDSSAAW
EQSLLEKFST ELYQQLNDLE
ACVIQEVGVE ETPLMNEDFI LAVRKYFQRI TLYLMEKKYS
PCAWEVVRAE IMRSFSFSTN
LKKGLRRKD
Human alpha-interferon (subtype 21):
MALSFSLLMA VLVLSYKSIC SLGCDLPQTH SLGNRRALIL
LAQMGRISPF SCLKDRHDFG
FPQEEFDGNQ FQKAQAISVL HEMIQQTFNL FSTKDSSATW
EQSLLEKFST ELNQQLNDLE
ACVIQEVGVE ETPLMNVDSI LAVKKYFQRI TLYLTEKKYS
PCAWEVVRAE IMRSFSLSKI
FQERLRRKE
Human alpha-interferon (subtype 10):
MALSFSLLMA VLVLSYKSIC SLGCDLPQTH SLGNRRALIL
LGQMGRISPF SCLKDRHDFR
IPQEEFDGNQ FQKAQAISVL HEMIQQTFNL FSTEDSSAAW
EQSLLEKFST ELYQQLNDLE
ACVIQEVGVE ETPLMNEDSI LAVRKYFQRI TLYLIERKYS
PCAWEVVRAE IMRSLSFSTN
LQKRLRRKD
Human alpha-interferon (subtype 14):
MALPFALMMA LVVLSCKSSC SLGCNLSQTH SLNNRRTLML
MAQMRRISPF SCLKDRHDFE
FPQEEFDGNQ FQKAQAISVL HEMMQQTFNL FSTKNSSAAW
DETLLEKFYI ELFQQMNDLE
ACVIQEVGVE ETPLMNEDSI LAVKKYFQRI TLYLMEKKYS
PCAWEVVRAE IMRSLSFSTN
LQKRLRRKD
Human alpha-interferon (subtype 5):
MALPFVLLMA LVVLNCKSIC SLGCDLPQTH SLSNRRTLMI
MAQMGRISPF SCLKDRHDFG
FPQEEFDGNQ FQKAQAISVL HEMIQQTFNL FSTKDSSATW
DETLLDKFYT ELYQQLNDLE
ACMMQEVGVE DTPLMNVDSI LTVRKYFQRI TLYLTEKKYS
PCAWEVVRAE IMRSFSLSAN
LQERLRRKE

An adjuvant, for example, one of polypeptide fragments of human alpha-interferon, may be used to obtain polyclonal antibodies to human alpha-interferon via immunization of rabbits.

Polyclonal antibodies to CD8 receptor are obtained following the method described above using adjuvant and entire molecule of CD8 receptor with the following amino-acid sequence as the immunogen (antigen) for rabbit immunization:

| | |
|---|---|
| 1 | MALVPVTALLL PLALLLHAAR PSQFRVSPLD RTWNLGETVE |
| | LKCQVLLSNP TSGCSWLFQP |
| 61 | RGAAASPTFL LYLSQNKPKA AEGLDTQRFS GKRLGDTFVL |
| | TLSDFRRENE GYYFCSALSN |
| 121 | SIMYFSHFVP VFLPAKPTTT PAPRPPTPAP TIASQPLSLR |
| | PEACRPAAGG AVHTRGLDFA |
| 181 | CDIYIWAPLA GTCGVLLLSL VITLYCNHRN RRRVCKCPRP |
| | VVKSGDKPSL SARYV. |

An adjuvant, for example, one of the fragments of CD8 receptor in accordance with the following sequences may be used to obtain polyclonal bodies to CD8 receptor via the immunization of rabbits:

11-30:
PLALLLHAAR PSQFRVSPLD;
81-100:
AEGLDTQRFS GKRLGDTFVL;
121-140:
SIMYFSHFVP VFLPAKPTTT;
201-210:
VITLYCNHRN;
221-235:
VVKSGDKPSL SARYV

Preferably, in order to prepare the medicinal agent, there is contemplated the usage of a mixture of three aqueous-alcohol solutions of the initial matrix solution of the antibodies diluted accordingly 100¹², 100³⁰ and 100²⁰⁰ times which corresponds to the centesimal solutions of C12, C30 and C 200 prepared by homeopathic technology. The mixture of dilutions is impregnated onto a neutral carrier, for the preparation of the claimed medicinal agent in solid form.

The activated-potentiated form of each component is prepared by uniform decrease of concentration as the result of consecutive dilution of 1 part of each solution that is to be diluted starting with the mentioned matrix solution, in 9 parts (for the decimal solution D) or 99 parts (for centesimal solution C) or 999 parts (for millesimal solution M) of the neutral solvent in combination with multiple vertical shaking (potentiation or “dinamization”) of each produced solution and the usage of separate containers for every further solution until the production of the required potency—the multiplicity of dilution in accordance with homeopathic technology (see, for example, W. Shwabe “Homeopathic medicinal preparations”, M., 1967, p. 14-29).

External treatment carried out together with the process of concentration decrease may also be effected by the use of ultrasound, electromagnetic or other physical action.

For example, for the preparation of 12^{th} centesimal dilution C12, one part of the matrix (primary) dilution of antibodies, for example, to the tumor necrosis factor-alpha with the concentration of 2.5 mg/ml is dissolved in 99 parts of neutral water or hydroalcoholic solvent and shaken vertically many times (10 or more times)—potentiate, obtaining first centesimal Cl dilution. From the 1^{st} centesimal dilution of C1 the 2^{nd} centesimal dilution of C2 is produced. The given operation is repeated for 11 times, producing 12^{th} centesimal dilution C12. Thus, the 12^{th} centesimal dilution C12 is a solution obtained by consequently diluting 12 times in different volumes the 1^{st} portion of the initial matrix solution antibodies to human gamma-interferon with the concentration of 2.5 mg/ml in 99 parts of the neutral carrier, i.e. the solution obtained by diluting matrix solution in 100¹² times. Analogous operations with the correspondent dilution multiplicity are executed in order to produce C30 and C50.

When using, for example, activated-potentiated form of antibodies to the tumor necrosis factor-alpha in the form of the mixture of different, predominantly centesimal, dilutions, each dilution (for example, C12, C30, C50) is prepared separately using the technology described above until obtaining a dilution which is three dilutions prior to the final dilution (correspondently, until producing C9, C27, C47) and thereafter introduce in accordance with expected content of the mixture into a single container one part of each component and mixing the mixture with the required amount of solvent (accordingly with 97 parts for centesimal dilution). Thereafter, the resulting mixture is consecutively diluted twice in the ratio of 1 to 100, potentiating the resulting solution after each dilution. As a result, there is obtained an activated-potentiated form of antibodies, for example, to human gamma-interferon in ultra-low dose, obtained by diluting the matrix solution in 100¹², 100³⁰ , 100⁵⁰ times, equivalent to the mixture of the centesimal solutions C12, C30, C50.

The use of each component separately is possible in the form of the mixture of other different dilutions, for example, decimal and/or centesimal, (D20, C30, C100 or C12, C30, C200 etc.), prepared with the use of homeopathic technology, whose effectiveness is determined experimentally.

To obtain solid dosage form of the claimed medicinal agent, granules of neutral carriers—lactose (milk-sugar) with particle sizes of 50-500 μm is impregnated with aqueous or aqueous-alcohol solutions of activated-potentiated form of antibodies to CD4 receptor in a fluid bed layered device (for example, “Hüttlin Pilotlab” type produced by the company of Hüttlin Gmbh); preferably in a ratio of 1 kg of solution of antibodies to 5 or 10 kg of lactose (1:5-1:10) with simultaneous drying in the flow of heated air at a temperature of not greater than 40° C. The calculated amount of lactose (10-91% of the tableting mass) impregnated with activated-potentiated form of antibodies in accordance with the above-described technology is loaded into a mixer and is mixed with lactose which is wetted with the activated-potentiated form of the antibodies, in the amounts of 3-10% of the tableting mass and with pure lactose in the amount of not more than 84% from the tableting mass (to reduce the cost and to obtain certain degree of simplification and accelerate the technological process without the reduction in effectiveness of medicinal treatment). Thereafter, in the same mixture, there is added microcrystalline cellulose in the amount of 5-10% of the tableting mass and magnesium stearate in the amount of 1% of the tableting mass. The produced tableting mass is uniformly mixed and tableted by dry compression (for example, in the tablet-press Korsch—XL 400). After the tableting, there are obtained tablets with the mass of 300 mg impregnated with aqueous alcohol solutions of the activated-potentiated form of antibodies to the tumor necrosis factor-alpha in ultra-low dose of each component prepared from the matrix solution dissolved in 100¹², 100³⁰, 100⁵⁰ which is equivalent to the mixture of centesimal dilutions C12, C30 and C50, prepared in accordance with homeopathic technology.

Preferably the claimed medicinal agent should be administered 3-4 times per day, 1-2 tablets each time.

### EXAMPLE 1

Antiretroviral action of the claimed medicinal agent was studied by inhibiting the replication of HIV in the culture of mononuclear cells of the peripheral human blood which were infected in vitro by the strain of HIV-1-LAI. Effective inhibition of HIV replication was evaluated by the content of basic nuclecapsid protein r24 in the supernatant of HIV.

For experimental studies researchers were used affinity purified rabbit polyclonal antibody to tumor necrosis factor alpha, prepared to order by dedicated biotechnology firm, which was the basis of preparation of activate-potentiated form aqueous dilutions of antibodies to tumor necrosis factor-alpha in the ultra-low dose obtained by homeopathic technology by ultradilution of initial matrix solution (concentration 2.5 mg/ml) in the 100¹², 100³⁰, 100⁵⁰ times, equivalent to the mixture of centesimal homeopathic dilutions C12, C30, C50 (hereinafter—ULD AB to TNF-alpha). Evaluation of the antiviral activity of complex preparation was carried out using mononuclear cells of peripheral blood, infected in vitro HIV-1-LAI.

Human peripheral blood mononuclear cells were isolated from the blood of healthy seronegative donors by density gradient centrifugation of Ficoll-Hypaque. Cells were activated for 3 days with 1 mg/ml phytogemmaglyutin P and 5 ME/ml of recombinant human interleukin-2

To assess the antiretroviral activity of drugs introduced into the well containing 100 μL of activated human peripheral blood mononuclear cells in 24 hours, or 15 minutes after infection of cells with HIV-1 strain-LAI at 100 TCID50 (50 μL of inoculate strain of HIV-1-LAI). Before introduction into the well, ULD AB to TNF-alpha (12.5 μL) or AZT (zidovudine, the active ingredient) in a dose of 1000 nM (the reference drug) were mixed with the medium RPMI1640 (DIFCO) to achieve a final volume of 50 μL.

Cell culture supernatants were collected at day 7 after infection. Efficacy of the drugs was determined by the inhibition of HIV replication, which was evaluated by content nucleocapsid core protein p24 of HIV in cell supernatants by ELISA (Retrotek Elisa kit).

It is shown that ULD AB to TNF alpha inhibits HIV replication by 92±3% upon entry into the well for 24 hours before and 13±13% upon entry into the well 15 minutes after infection of cells with HIV-1 strain-LAI, respectively. AZT at a dose of 1000 nM inhibited the replication of HIV by 99±0 and 99±1% at entry to the well for 24 hours before and 15 minutes after infection of cells strain of HIV-1-LAI, respectively.

Thus, the in vitro demonstrated high antiretroviral activity claimed preparation of activated-potentiated form rabbit polyclonal antibodies to TNF-alpha.

Note: TCID50 is the dosage that infects 50% of the tissue culture cells.

### EXAMPLE 2

Antiretroviral effect of the claimed medicinal agent studied by inhibiting HIV replication in cultured peripheral blood mononuclear cells of human infected in vitro HIV-1-LAI. Effective inhibition of HIV replication was evaluated by the content of basic nucleocapsid protein p24 in the supernatants of HIV.

For experimental studies the researchers used affinity purified rabbit polyclonal antibody to CD8, prepared to order by dedicated biotechnology firms, on the basis of which was prepared to activate—potentiated form aqueous dilutions of antibodies to CD8 in midget doses received by homeopathic technology by the super-dilution of the source matrix solution (concentration of 2, 5 mg/ml) in the 100¹², 100³⁰, 100⁵⁰ times, hundred-equivalent mixture of homeopathic dilutions from C12, C30, C50 (hereinafter ULD AB to CD8)

Evaluation of the antiviral activity of complex preparation was carried out using mononuclear cells of peripheral blood, infected in vitro HIV-1-LAI.

Human peripheral blood mononuclear cells were isolated from the blood of healthy seronegative donors by density gradient centrifugation of Ficoll-Hypaque. Cells were activated for 3 days with 1 mg/ml phytohemaglyutin P and 5 ME/ml of recombinant human interleukin-2

To assess the antiretroviral activity of drugs introduced into the well containing 100 μL of activated human peripheral blood mononuclear cells in 24 hours, or 15 minutes after infection of cells with HIV-1 strain-LAI at 100 TCID50 (50 μL of inoculate strain of HIV-1-LAI). Before introduction into the well, ULD AB to CD8 (12.5 μL) or AZT (zidovudine, the active ingredient) in a dose of 1000 nM (reference drug) were mixed with the medium RPMI1640 (DIFCO) to achieve a final volume of 50 μL.

Cell culture supernatants were collected at day 7 after infection. Efficacy of the drugs was determined by the inhibition of HIV replication, which was evaluated by content nucleocapsid core protein p24 of HIV in cell supernatants by ELISA (Retrotek Elisa kit).

It is shown that ULD AB CD8 inhibits HIV replication by 87±11% at entry to the well 24 hours before and 40±4%, with introduction into the well 15 minutes after infection of cells by strain of HIV-1-LAI, respectively. AZT at a dose of 1000 nM inhibited the replication of HIV by 99±0 and 99±1% at entry to the well for 24 hours before and 15 minutes after infection of cells strain of HIV-1-LAI, respectively

Thus, the in vitro study showed high antiretroviral activity of ultra low doses of rabbit polyclonal antibody to CD8

Note: TCID50—dosage, infecting 50% of the tissue culture cells.

### EXAMPLE 3

Estimation of antiretroviral activity of activated-potentiated forms of water dilutions of polyclonal affinity purified rabbit antibodies to interferon alpha in ultralow doses (ULD) received by superdilution of the source matrix solution (2.5 mg/ml) in 100¹², 100³⁰ , 100⁵⁰ time equivalent mixture of homeopathic centesimal dilutions with C12, C30, C50 (ULD AB to IFN-alpha) was carried out using mononuclear cells of peripheral blood, infected in vitro HIV-1-LAI. AZT was used as a reference drug.

Human peripheral blood mononuclear cells were isolated from the blood of healthy seronegative donors by density gradient centrifugation of Ficoll-Hypaque. Cells were activated for 3 days with 1 mg/ml phytogemmaglyutin P and 5 ME/ml of recombinant human interleukin-2.

Cells were infected with HIV-1-LAI, making 50 μL inoculate strain of HIV-1-LAI, which corresponds to a dose of 100 TCID50 (dose infecting 50% tissue culture cells).

To assess the antiretroviral activity of the drug ULD AB to IFN-alpha and the reference drug AZT were added into wells containing 100 μL of activated human peripheral blood mononuclear cells, 24 hours before and 15 minutes after infection of cells strain of HIV-1-LAI. Before introduction into the wells, the drug ULD AB to IFN-alpha (12.5 μL) and the drug AZT at 1,000 nM were mixed with the medium RPMI1640 (DIFCO) to achieve a final volume of 50 μL.

Cell culture supernatants were collected at day 7 after infection. Efficacy of the drugs was determined by the inhibition of HIV replication, which was estimated by the content of basic nucleocapsid protein of HIV p24 in cell supernatants by ELISA (Retrotek Elisa kit).

It is shown that the drug ULD AB to IFN-alpha inhibits the replication of HIV by 95±2% at entry to the well for 24 hours before and 59±14% upon entry into the well 15 minutes after infection of cells strain of HIV-1-LAI, respectively. AZT at a dose of 1000 nM inhibits the replication of HIV by 99±0 and 99±1% at entry to the well for 24 hours before and 15 minutes after infection of cells strain of HIV-1-LAI, respectively.

Thus, in the in vitro study showed high activity antiretroviral drug ULD AB to IFN-alpha.

1.-19. (canceled)

20. A medicinal agent comprising an activated-potentiated form of antibodies to a protein or peptide of the immune system which reacts with HIV or whose content and/or functional activity varies in relation to HIV infection.

21. The medicinal agent as defined in claim 20, characterized in that the activated-potentiated form of antibodies is to a dissolved antigen.

22. The medicinal agent as defined in claim 21, characterized in that the dissolved antigen is a cytokine, except for IFN-gamma.

23. The medicinal agent as defined in claim 20, characterized in that the activated-potentiated form of antibodies is to an antigen associated with the outer membrane of cells of the immune system.

24. The medicinal agent as defined in claim 23, characterized in that the antigen associated with the outer membrane of cells of the immune system, is receptors of immune cells.

25. The medicinal agent as defined in claim 23, characterized in that the antigen associated with the outer membrane of cells of the immune system, is clusters of differentiation, except for the CD4 molecule of T lymphocytes.

26. A pharmaceutical composition comprising the medicinal agent as defined in claim 20, characterized in that the activated-potentiated form of antibodies is in the form of activated-potentiated water or a water-alcohol solution, which is obtained by the process of multiple consecutive dilutions of multiple source matrix in combination with an external mechanical action, vertical shaking each dilution.

27. The pharmaceutical composition as defined in claim 26, characterized in that the medicinal agent is in the form of solid dosage, which contains the technologically necessary amount of neutral carrier impregnated with mixture of the aqueous or aqueous-alcoholic solutions of the activated potentiated form of antibodies and pharmaceutically acceptable excipients.

28. The pharmaceutical composition as defined in claim 26, characterized in that the aqueous or aqueous-alcoholic solutions of the activated-potentiated forms antibodies is obtained by multiple consecutive dilutions of the initial matrix solution of antibodies in combination with external mechanical action—vertical shaking of each dilution, the concentration of matrix solution being 0.5-5.0 mg/ml.

29. The pharmaceutical composition as defined in claim 28, characterized in that the activated-potentiated form antibodies used is a mixture of different dilutions obtained by multiple consecutive dilutions of the initial matrix solution of antibodies in combination with external mechanical action—vertical shaking of each dilution.

30. The pharmaceutical composition as defined in claim 29, characterized in that the activated-potentiated form antibodies used is a mixture of centesimal of dilutions obtained by multiple consecutive dilutions of the initial matrix solution of antibodies in combination with external mechanical action—vertical shaking of each dilution.

31. The pharmaceutical composition according to claim 27, characterized in that the pharmaceutically acceptable excipients include lactose, microcrystalline cellulose, and magnesium stearate.

32. A method of prophylaxis of HIV infection, prophylaxis and treatment of diseases caused by HIV or associated with HIV, including AIDS, said method comprising administering to a subject in need thereof a medicinal agent comprising an activated-potentiated form of antibodies to the antigen-protein or peptide of the immune system, which interacts with HIV or whose content and/or functional activity varies in relation to HIV infection.

33. The method according to claim 32, characterized in that the activated-potentiated form antibodies, are used as activated-potentiated water or a water-alcohol solution obtained by multiple-source dilution matrix—the antibody solution in an aqueous or aqueous—alcoholic solvent in combination with an external mechanical action, vertical shaking each dilution.

34. The method according to claim 33, characterized in that the water or water-alcohol solutions activated-potentiated forms of antibodies are obtained by multiple consecutive dilution of the initial matrix solution, the antibody solution in conjunction with an external mechanical action—vertical shaking each dilution the concentration of the matrix solution of 0.5-5.0 mg/ml.

35. The method according to claim 32, characterized in that the activated-potentiated form antibodies is to a dissolved antigen.

36. The method according to claim 32, characterized in that the dissolved antigen is cytokines, except for IFN-gamma.

37. The method according to claim 32, characterized in that the activated-potentiated form antibodies is to an antigen associated with the outer membrane of cells of the immune system.

38. The method according to claim 32 characterized in that the antigen associated with the outer membrane of cells of the immune system is receptors of immune cells.

39. The method according to claim 32, characterized in that the antigen associated with the outer membrane of cells of the immune system, is clusters of differentiation, with the exception of CD4 molecules of T lymphocytes.
